(19) United States
(12) Patent Application Publication
(10) Pub. No.: US 2021/0030647 A1
(43) Pub. Date: Feb. 4, 2021

(54) REGENERATING COMPOSITION WITH SMOOTHING ACTION FOR TREATING SKIN IMPERFECTIONS AND PROCESS FOR PREPARATION THEREOF

(71) Applicant: PROMOITALIA GROUP S.P.A., Milano (IT)

(72) Inventors: Egidio TRANFAGLIA, Napoli (IT); Valerio MATANO', Napoli (IT)

(21) Appl. No.: 16/967,050

(22) PCT Filed: Sep. 12, 2018
(86) PCT No.: PCT/IB2018/056958

§ 371 Date: Aug. 3, 2020

(30) Foreign Application Priority Data
Sep. 12, 2017 (IT) 102017000102009

(51) Int. Cl. A61K 8/365; A61K 8/34; A61K 8/66; A61K 8/41; A61K 8/73; A61K 8/97; A61K 8/49; A61K 8/22; A61Q 19/08

(52) U.S. Cl. CPC A61K 8/365; A61K 8/345; A61K 8/66; A61K 8/41; A61K 8/73; A61K 2800/805; A61K 8/498; A61K 8/22; A61Q 19/08; A61K 2800/59; A61K 8/97

(57) ABSTRACT

Disclosed is a regenerating composition having skin lifting and anti-age effect based on chlorinated derivatives of acetic acid, propylene glycol and sodium hydroxide, for use in the treatment of skin blemishes caused by stretch marks, acne scars, sun damage, skin sagging, hyperpigmentation, scars in various parts of the body, such as face, neck, chest, breast, inner thighs, arms, mons pubis, labia majora, inguinal and perianal areas, which is capable of overcoming the problems which are still present in the products of the prior art, a process of preparing the composition, and a method of use.

### FIELD OF APPLICATION

The present invention relates to a skin treatment product for cosmetic purposes; in particular, it relates to a composition for use in regenerating treatments with skin lifting and anti-age effect, for treating skin blemishes caused by stretch marks, acne scars, sun damage, skin sagging, hyperpigmentation, scars in various parts of the body, such as face, neck, chest, breast, inner thighs, arms, mons pubis, labia majora, inguinal and perianal areas.

### Prior Art

Many problems related to the appearance of the skin as a result of acne scars and to facial rejuvenation are currently treated with the aid of new technologies. Topical products which are the most appropriate and safest for local use and systemic antioxidants which can contrast the action of the free radicals responsible for damaging the DNA structure of cells are being studied based on skin type and condition analysis.

One of the most commonly used methods is chemical peeling, which is a cosmetic treatment stimulating exfoliation and consequent skin cell renewal by applying a chemical substance to the skin.

Chemical peeling acts on the skin by means of various mechanisms:
it stimulates cell turnover by removing and exfoliating dead cells of the stratum corneum, thus genuinely boosting the cell renewal which already naturally takes place in the dermis;
it eliminates damaged and degenerated skin cells, which are replaced by normal skin cells;
it produces a temporary local inflammation which activates the production of collagen and elastin (to revitalize and rejuvenate the dermis by stimulating fibroblasts with formation of autologous collagen).

Therefore, peeling may act on both the more superficial and the deeper levels of the dermis, but deep peeling implies a greater risk of side effects; therefore, it is preferable to perform multiple superficial or at most medium depth peeling sessions.

Peeling is currently widely used in dermatology, in which new substances, such as mandelic acid (an alpha-hydroxy acid having high molecular weight), pyruvic acid, kojic acid, salicylic acid or associations of multiple acids are being used, in addition to simple glycolic acid, which has been popular since the early 1990s. Indeed, so-called peeling compounds, i.e. combinations of multiple substances in the same product, which utilize the synergetic action of the various substances, are used.

One of the most effective chemical peeling agents is certainly trichloroacetic acid (TCA). Trichloroacetic acid peeling always requires full prior awareness of its potentials and its limitations because the method is not free from risks and side effects, such as skin maceration and necrosis. The treatment may be more or less aggressive according to the percentage of acid used. It is used in 10-25% solution for superficial peeling, to contrast early signs of aging (thin wrinkles, dull complexion), acne, superficial keratoses, for deep skin cleansing and to shrink pores. TCA at 35-50%, instead, produces a much deeper exfoliation, which reaches the deeper layers of the skin (the reticular dermis) and often forces the patient to stay indoors for 6-7 days; in all cases, effects are optimum and skin is completely renewed.

Trichloroacetic acid does not require neutralization once applied; it may be washed with water to cool and soothe burning.

After peeling with TCA, the skin will have a brown complexion, which will be replaced, after exfoliation, by a slight redness which will disappear within 24-48 hours. During this exfoliation period, which may last for up to one week (according to the concentration used), solar exposure must be avoided and the subjects must not pick skin residues; healing times are of the order of a few days for light peeling (10% TCA) and of about 7-10 days for deeper peeling (TCA 25-35%).

The side effects and drawbacks of chemical peeling performed with TCA can be considerable, because the agent is rather aggressive and in order to achieve the technical effect based on the benefits of exfoliation and skin renewal it must penetrate in depth, crossing the epidermis to the dermis, thus causing structural damage to the epidermis and dermis as a result of denaturation and skin protein precipitation, which typically causes a whitening of surface skin. Protein denaturation produces an eschar, i.e. a dark skin plaque of narcotized tissue, the healing of which will result in the desired skin renewal; however, the healing and replacement process takes from 7 to 10 days, during which the subject's skin will appeared covered by an unattractive, thick dark scab.

Italian patent no. 1359709 describes a product for treating skin pathologies, such as acne, scarring caused by acne, chicken pox and the like, sun damage and aging, freckles, wrinkles, skin sagging and the like, comprising an appropriate amount of trichloroacetic acid ranging between 15 and 50% by weight, preferably equal to 33% by weight, and an appropriate amount of hydrogen peroxide ranging between 85 and 50% by weight, preferably equal to 67% by weight, and an appropriate amount of basic compound chosen from ammonia, triethanolamine and the like, having a pH between 2.3 and 2.6, adapted to be administered to a patient to perform the cosmetic skin treatment.

In order to alleviate the side effects of the compositions used for TCA-based cosmetic treatments, products have been developed and marketed, in which 5% kojic acid is also used in association with 33% trichloroacetic acid and hydrogen peroxide, which kojic acid quickly penetrates in keratinocyte cell membranes and immediately neutralizes the excessive exfoliating effect of the TCA.

However, the traumatic event for skin which must trigger a restructuring action and a genuine bio-revitalizing process underlies the sought technical effect of the regenerative and lifting action.

Despite the use of solutions aimed at limiting or eliminating the many, obvious disadvantages of the TCA-based chemical peeling methods, these cannot overcome the problems still present in the prior art; therefore, the need is strongly felt to have alternative products based on TCA exfoliating agent which can maximize the lifting and bio-regenerating effects of peeling, thus canceling out the side effects.

### SUMMARY OF THE INVENTION

It is the object of the present invention to provide a regenerating composition with skin lifting and anti-age effect for use in the treatment of skin blemishes caused by stretch marks, acne scars, sun damage, skin sagging, hyperpigmentation, scars in various parts of the body, such as face, neck, chest, breast, inner thighs, arms, mons pubis, labia majora, inguinal and perianal areas, which is capable of overcoming the problems which are still present in the products of the prior art.

The present invention further relates to the process of preparing said composition and to the method of use thereof.

### DETAILED DESCRIPTION OF THE INVENTION

The described object is achieved by using a composition to be used in chemical peeling which can maximize the lifting and bio-regenerating effects of the chemical peeling, limiting disadvantages and side effects for the patient.

The composition according to the invention comprises a mixture of one or more chlorinated derivatives of acetic acid, glycolic solvent, preferably propylene glycol, buffered with a basic compound, to obtain a final pH value of the composition between 2.0 and 4.0. Preferably the pH value of the composition is between 2.0 and 3.0.

According to the invention, the chlorinated derivatives of acetic acid are preferably monochloroacetic acid (MCA), dichloroacetic acid (DCA) and trichloroacetic acid (TCA).

The composition according to the invention is therefore characterized, as compared to the compositions of the prior art based on TCA and hydrogen peroxide, by the presence of propylene glycol solvent in association with one or more chlorinated derivatives of acetic acid.

The composition according to the present invention, being based on one or more chlorinated derivatives of acetic acid, dissolved in glycolic solvent, at controlled pH, has greater penetrative capacity than that of the prior art, because such a solvent can reduce the polarity of the aforesaid molecules; so, the chlorinated derivative of acetic acid, which acts as an exfoliating agent, solvated in glycol, can quickly reach the medium-deep dermis layer, where the amount of aqueous solution present in the patient's ground substance is greater than the glycogen composition. Having reached the dermis, the chlorinated derivative of acetic acid undergoes a solvent modification, tends to solubilize in the aqueous component present in the dermis, thus becoming more acid and thus more capable of attacking the dermal proteins and collagen present in the skin layer, which is the true target of the renewing action. By means of the composition according to the invention, an effective protein denaturation is thus obtained, capable of stimulating the production of new fibers by the fibroblasts without using the pro-oxidant substances, such as hydrogen peroxide; the composition thus explicates its regenerating and anti-aging effect by limiting the side effects of the chemical peeling.

According to the invention, the preferred buffering agent is sodium hydroxide. The use of sodium hydroxide as a buffering agent in the glycolic environment confers particularly advantageous properties to the composition according to the invention as compared to the compositions of the prior art which employ ammonia derivatives or triethanolamine (TEA) for the same purpose. Indeed, the use of ammonia derivatives or triethanolamine has the drawback of making the composition less stable because the salt resulting from neutralization can easily degrade, particularly if in association with oxidizing substances, such as hydrogen peroxide, or functional equivalents thereof.

Therefore, the composition according to the invention comprising the mixture of one or more chlorinated derivatives of acetic acid, in glycolic solvent, buffered with sodium hydroxide, instead of ammonia derivatives or TEA, is particularly stable; this avoids the possible production of toxic metabolites resulting from the degradation of the active principles a posteriori.

Indeed, in the compositions of the prior art the hydrogen peroxide, which is very reactive on cellular structures, in acid environment, tends to decompose very easily into water and gaseous oxygen, thus exposing the product to risk of explosion, among other risks. The mixture thus degraded can no longer be used, because the chlorinated acetic acid is not appropriately buffered, and this exposes the patient to the possible risk of white frost, which is the typical superficial whitening effect due to denaturation and precipitation of dermal proteins upon an excessively aggressive peeling.

Furthermore, because of their high polarity, peeling mixtures based on TCA and hydrogen peroxide have a lower penetrating capacity with consequent effect limited only to the surface layers of the dermis.

Ammonia derivatives or TEA have the drawback of making the compound less stable because the salt deriving from neutralization degrades more easily, especially if in association with oxidizing substances, such as hydrogen peroxide or derivatives. For this reason, it is preferable to store cosmetic products containing such mixtures at lower temperatures than ambient temperatures.

These drawbacks are all absent in the composition according to the present invention which is free from hydrogen peroxide, or its equivalents, and uses sodium hydroxide in glycolic environment for controlling the pH value.

The large progress over recent years in the characterization of the biological processes underlying the aging process in terms of appearance and function, with the consequent identification of the best and most effective strategies to treat the effects of such an aging, have shown how aging is the result of a complex set of biological and metabolic processes and is greatly influenced by genetics. Therefore, it has been observed that for a more comprehensive and effective approach to contrasting the effects of skin aging, it is useful to combine an antioxidant action, anti-wrinkle/anti-age and lightening effect with the specific dermo-cosmetic treatment action provided by the chemical peeling in order to provide the basic elements and molecules for regenerating and maintaining a healthy skin.

According to another aspect of the invention, in the described composition, the treating effect of the mixture of chlorinated derivatives of acetic acid and propylene glycol, buffered by sodium hydroxide, may be further boosted by the action of other molecules which form part of the formulation of the present composition as components with accessory function, such as for example: urea peroxide, coenzyme Q10, mandelic acid, kojic acid, hexapeptide-1, Glycyrrhiza glabra extract.

Urea peroxide breaks down into oxygen, which results in an oxidizing and whitening effect, and into urea, which acts as a moisturizer and keratinolytic agent. Fifty percent of the peroxide breaks down into oxygen during the first 2 hours after application and remains active for other 6 hours, thus causing a prolonged effect over time.

Coenzyme Q10 or ubiquinone is a hydrophilic/lipophilic balance molecule such as to ensure action both in aqueous and hydrophobic environment. Its role consists in acting as a scavenger of the free radical present both locally and resulting from urea peroxide dissociation.

Mandelic acid belongs to the category of alpha-hydroxy acids, which are molecules widely used in cosmetics precisely for producing exfoliating, lightening and antioxidizing creams.

Mandelic acid is characterized and widely applied for its extraordinary exfoliating properties and its use in cosmetic products offers considerable advantages as compared to other alpha-hydroxy acids. Unlike glycolic creams and products prepared with other chemical exfoliants, cosmetics based on mandelic acid are not photosensitizers. Furthermore, mandelic acid—once applied to the skin—produces less erythema, which in some cases is nearly imperceptible.

Kojic acid present in the composition according to the invention provides a lightening activity and has depigmentation (as well as antioxidizing) properties. The kojic acid molecule acts by penetrating in depth into the skin layers where it blocks the action of enzymes which are involved in the melanin production process; this determines the positive effect in the treatment of the signs of skin aging, as well as in the treatment of hyperpigmentation on both surface and deep levels, where it is effective by acting on stratum corneum level to contrast the signs of aging in the skin and to stimulate and revitalize skin elasticity.

The effect of other secondary blemishes which are typical of skin aging is contrasted by the action of hexapeptide-1 present in the composition according to the invention. Indeed, it is known that the contractions of facial expression muscles are a fundamental stimulus for the formation of wrinkles caused by both normal aging progression and negative environmental factors (solar radiation). The final result is a series of metabolic, cellular, histological and physiological processes which culminate in the loss of elasticity, water, number of elastic fibers by the dermal and epidermal stromal cells and the production of free radicals. The hexapeptide-1 used in the present composition is a peptide consisting of six amino acids, having activity comparable to that of botulinum toxin A, acting on the release of catecholamines, which are the chemical mediators of the muscle contraction; therefore, one applied to the skin it can temporarily reduce the tonic contraction of facial muscles responsible for the appearance of expression wrinkles. It is used in skin firming and anti-age products for its ability to distend deep creases and expression wrinkles.

Glycyrrhiza glabra extract has a major anti-inflammatory, protective and stabilizing action on cell membranes.

Therefore, by virtue of a unique synergy of active components absorbed through the skin, longer lasting and tangible results against the signs of aging on the treated skin are ensured; advantageously, the composition according to the invention as a chemical peeling agent combines powerful dermal and epidermal cell and tissue renewal properties due to components having antioxidizing, anti-wrinkle-forming/anti-aging and lightening activities, without causing particularly severe side effects in the treated body region, e.g. exfoliation resulting from dermal and epidermal protein denaturation, as indicated by the skin lightening effect or the release of toxic products resulting from the degradation of the active principles used.

In a first embodiment, the composition according to the invention comprises:
a mixture of one or more chlorinated derivatives of acetic acid, in a concentration ranging between 1 and 50% by weight of total composition, preferably between 25 and 35% by weight of total composition;
glycerol, in a concentration ranging between 1 and 20% by weight of total composition, preferably between 5 and 15% by weight of total composition;
glycolic solvent in a concentration ranging between 1 and 10% by weight of total composition, preferably between 3 and 8% by weight of total composition, wherein the glycolic solvent is preferably propylene glycol;
mandelic acid in a concentration ranging between 1 and 10% by weight of total composition; preferably between 3 and 8% by weight of total composition;
kojic acid in a concentration ranging between 1 and 10% by weight of total composition; preferably between 3 and 8% by weight of total composition;
sodium hydroxide in a concentration ranging between 1 and 10% by weight of total composition;
preferably between 0.5 and 1.5% by weight of total composition;
xanthan gum, in a concentration ranging between 0.1 and 3% by weight of total composition, preferably between 0.25 and 2% by weight of total composition;
hexapeptide-1, in a concentration ranging between 0.1 and 5% by weight of total composition, preferably between 1.5 and 3.0% by weight of total composition;
coenzyme Q10, in a concentration ranging between 0.01 and 2% by weight of total composition, preferably between 0.1 and 1% by weight of total composition;
distilled water up to 100% by weight.

In a second embodiment, the composition according to the invention comprises:
urea peroxide in a concentration ranging between 0.1 and 0.09%.

With reference to the first embodiment of the invention, a particularly preferred formulation comprises:
a chlorinated derivative of acetic acid, selected from monochloroacetate and dichloroacetate, 33% by weight of the total composition;
propylene glycol, 5% by weight of total composition;
Glycyrrhiza glabra root extract, 0.1% by weight of the total composition;
mandelic acid, 5% by weight of total composition;
kojic acid, 5% by weight of total composition;
sodium hydroxide, 1% by weight of total composition;
xanthan gum, 0.5% by weight of total composition;
hexapeptide-1, 2.5% by weight of total composition;
coenzyme Q10, 0.2% by weight of total composition;
urea peroxide, 1% by weight of total composition;
distilled water up to 100% by weight.

With reference to the second embodiment of the invention, a particularly preferred formulation comprises:
trichloroacetic, 33% by weight of total composition;
glycerol, 10% by weight of total composition;
propylene glycol, 5% by weight of total composition;
mandelic acid, 5% by weight of total composition;
kojic acid, 5% by weight of total composition;
sodium hydroxide, 1% by weight of total composition;
xanthan gum, 0.5% by weight of total composition;
hexapeptide-1, 2.5% by weight of total composition;
coenzyme Q10, 0.2% by weight of total composition;
distilled water up to 100% by weight.

The compositions according to the invention further comprise excipients known to those skilled in the art, such as viscosifying agents, preservatives, antioxidants.

The compositions according to the invention are presented in a suitable form for topical administration to the skin of the patient who is the recipient of the treatment aimed at eliminating or reducing skin blemishes caused by stretch marks, acne scars, sun damage, skin sagging, hyperpigmentation, scars in various parts of the body, such as face, neck, chest, breast, inner thighs, arms, mons pubis, labia majora, inguinal and perianal areas, such as for example cream, gel, ointment, liquid solution, patch.

The method of using the compositions according to the invention includes the following protocol for each single application:
a) thoroughly cleansing and drying the skin of the area where the treatment is to be administered;
b) applying an amount of composition adapted to cover an area of about 5-10 cm² of skin surface and massaging until full absorption;
c) proceeding as described in b) until the entire area is treated;
d) repeating steps b) and c) up to a maximum of five times in the same treatment session in the same skin areas;
e) applying a nourishing, sunscreen cream to the entire treated area.

The described treatment method can be repeated after 15 days for up to 6 sessions.

Such a treatment cycle may be performed twice a year after 6 months.

The opportunity to subject the patient to the described treatment must be assessed by a doctor.

If the chlorinated derivative of acetic acid is a monochloroacetic or dichloroacetic acid, and mixtures thereof, the respective composition can be obtained by means of a preparation process which comprises the following steps:
a)—preparing a mixture of an appropriate amount of mandelic acid, an appropriate amount of monochloroacetic and/or trichloroacetic acid and an appropriate amount of kojic acid in water;
b)—preparing a second mixture obtained by dissolving the appropriate amount of urea peroxide in glycolic solvent, where the solvent is preferably propylene glycol;
c)—mixing the mixture obtained in a) with the mixture obtained in b);
d)—slowly adding to the mixture obtained in c) an amount of sodium hydroxide adapted to allow the mixture achieving a final pH value between 2.0 and 4.0, preferably between 2.0 and 3.0;
e)—adding to the mixture obtained in d) all the remaining components one by one and adjusting the final volume with distilled water.

Instead, if the chlorinated derivative of acetic acid is TCA, the process involves:
a) preparing a mixture of an appropriate amount of mandelic acid, an appropriate amount of trichloroacetic acid and an appropriate amount of kojic acid in water;
b) adding to the mixture obtained in a) an appropriate amount of glycolic solvent, wherein the solvent is preferably propylene glycol;
c) slowly adding to the mixture obtained in b) an amount of sodium hydroxide adapted to allow the mixture achieving a pH value between 2.0 and 4.0, preferably between 2.0 and 3.0.
d) adding to the mixture obtained in c) all the remaining components one by one and adjusting the final volume with distilled water.

The appropriate amount of mandelic acid used in the process of preparing the compositions according to the invention is that allowing a final composition to be obtained, in which the concentration of mandelic acid is between 1 and 10% by weight of total composition; preferably between 3 and 8% by weight of total composition; the appropriate amount of chlorinated derivative of acetic acid used in the process of preparing the compositions according to the invention is that allowing a final composition to be obtained, in which the concentration of chlorinated derivative of acetic acid is between 1 and 50% by weight, preferably between 25 and 35% by weight; where the concentration of mandelic acid is between 10 and 50% by weight, preferably between 25 and 35% by weight; the appropriate amount of kojic acid used in the process of preparing the compositions according to the invention is that allowing a final composition to be obtained, in which the concentration of kojic acid is between 1 and 10% by weight of total composition; preferably between 3 and 8% by weight of total composition; the appropriate amount of propylene glycol used in the process of preparing the compositions according to the invention is that allowing a final composition to be obtained, in which the concentration of propylene glycol is between 1 and 10% by weight of total composition; preferably between 3 and 8% by weight of total composition.

It is to be understood that all the remaining components of the composition are added to the mixture obtained in d) in an amount adapted to obtain the final concentration of each one in the composition described above.

In step d) of the two described processes, sodium hydroxide is added so that it will not cause the increase of the temperature of the mixture being formed without resorting to external cooling means.

The invention will now be described by means of formulation examples and experiments in which the present invention was used, which allow highlighting the advantage resulting from its use as compared to the compositions of the prior art used for the same purpose.

### EXAMPLES

### Example 1—Formulation A

| Ingredient | % w/w |
|---|---|
| Trichloroacetic acid | 33 |
| Glycerol | 10 |
| Propylene glycol | 5 |
| Mandelic acid | 5 |
| Kojic acid | 5 |
| Xanthan gum | 0.5 |
| Sodium hydroxide | 1 |
| Hexapeptide-1 | 2.5 |
| Coenzyme Q10 | 0.2 |
| Distilled water | q.s. to 100 |

### Example 2—Formulation C

| Ingredient | % w/w |
|---|---|
| Monochloroacetic acid | 33 |
| Urea peroxide | 1 |
| Propylene glycol | 5 |
| Glycyrrhiza glabra root extract | 0.1 |
| Kojic acid | 5 |
| Xanthan gum | 0.5 |
| Sodium hydroxide | 1 |
| Hexapeptide-1 | 2.5 |
| Coenzyme Q10 | 0.2 |
| Distilled water | q.s. to 100 |

Experimental Part

It is the object of the trial to assess the effectiveness of the composition according to the invention characterized in that it comprises a mixture of chlorinated acetic acid, in glycolic solvent, buffered with sodium hydroxide for use in treating skin blemishes due to aging, the presence of stretch marks, acne or acne scars, generalized laxity, comparing it with compositions characterized in that they contain a mixture of chlorinated derivative of acetic acid, hydrogen peroxide or urea, buffered with a basic compound such as ammonia or TEA as an exfoliating agent.

Exclusion criteria in the trial were:
history of acute allergic reactions;
women of childbearing age who were not using contraceptives;
somatic disease, i.e. a disease or condition which, according the researcher, would make it difficult to interpret the test result;
HIV infection;
pregnancy and breastfeeding;
suspected malignant neoplasia;
symptoms due to altered mental state or capacity (psychosis, delusions, hallucinations);
patients with a history of drug addiction or constant alcohol abuse, which could adversely affect the patient's compliance with the trial procedures;
participation in another clinical trial during the last 30 days prior to the beginning of this trial.

The following patients were included in the trial:
patients with non-uniform skin elasticity and cutaneous pigmentations;
patients with degenerative alterations of the dermis due to age, skin and subcutaneous fat, which occur in the form of several wrinkles
patients with stretch marks
patients with acne and acne scars
patients with vaginal and perianal area laxity.

Further details are provided in the final table.

The experimentation was conducted on a sample of 16 patients divided into four experimental groups: Groups A, B, C and D.

All patients enrolled in the trial, of 25-65 years of age, were informed on the type of treatment, signed an informed consent and voluntarily subjected to the treatment, which was administered according to the methods and dosages provided for by the method described in the present document.

The patents in Group A received Formulation A of the composition shown in Example 1 above; the patents in Group C received Formulation C of the composition shown in Example 2 above; the patients in Group B received pharmaceutical Formulation B comprising: 33% trichloroacetic acid, 5% kojic acid, hydrogen peroxide and stabilizing agents.

The patients in Group D received galenic Formulation D comprising: 33% monochloroacetic acid, 5% kojic acid, hydrogen peroxide and stabilizing agents.

The efficacy of the compositions of Formulations A and C according to the invention in the cosmetic treatment aimed at reducing or eliminating skin blemishes were compared with compositions B and D used in chemical peeling practice.

Table 1 below summarizes the characteristics of the experimental groups and the type of treatment.

TABLE 1

| | Type of treatment | |
|---|---|---|
| | (administered | Number of |
| | composition) | subjects |
| Group A | Formulation A (TCA, | 4 |
| | propylene glycol, | |
| | sodium hydroxide) | |
| Group B | Formulation B (TCA, | 4 |
| | hydrogen peroxide, | |
| | TEA) | |
| Group C | Formulation C (MCA, | 4 |
| | propylene glycol, | |
| | sodium hydroxide) | |
| Group D | Formulation D (MCA, | 4 |
| | hydrogen peroxide, | |
| | TEA) | |

Before the first treatment session, the patients were subjected to a detailed analysis consisting of naked eye examination, followed by analysis using a magnifying glass under artificial white light (neon light) source and under Wood lamp, and then examination of the blemishes by palpation. The area(s) to be treated were analyzed by touch to assess the friction and tactile feeling of any irregularities and skin compactness, with pinching maneuvers to determine the mechanical properties of distensibility and elasticity.

Photographs of the areas to be treated were taken under the same conditions to obtain an objective assessment of the results obtained at a distance of time during the treatment and the follow-up.

Effectiveness, safety and tolerability of the treatment were assessed by using an arbitrary scale of levels of improvement of the typical clinical signs of skin alteration specially formulated by the trial experimenters, which took into account the results of the initial analysis performed on the patients compared with the signs present on patients after a one-year period from the beginning of the trial during which the patient received two treatment cycles, of six administrations each, six months apart.

TABLE 2
Scale of levels of improvement of clinical signs

| Level | Result obtained | Effect obtained |
|---|---|---|
| 1 | Excellent | Exceptional correction |
| | improvement | after one treatment |
| | | session |
| 2 | Considerable | Significant but not yet |
| | improvement | optimal improvement of |
| | | appearance |
| 3 | Improvement | Improvement of appearance |
| | | with respect to initial |
| | | condition, it is |
| | | advisable to continue the |
| | | treatment |
| 4 | No change | No change of appearance |
| | | with respect to initial |
| | | condition |
| 5 | Worsening | Worsening of appearance |
| | | with respect to initial |
| | | condition |

In the scope of the present trial, in order to achieve a correct, possibly objective assessment of the effectiveness, the term “side effect” means a non-intentional effect which occurs at the doses normally used and which is connected to the properties of the product, i.e. any unexpected or undesired, not necessarily harmful effect linked to the action of the administered composition; the term “adverse reaction” means an unwanted, harmful effect upon the use of the administered composition; instead, the term “adverse event” means any unpleasant clinical phenomenon which occurs during a treatment with the administered composition but without necessarily having a cause-effect link or relationship with the treatment itself. Therefore, the adverse event could be caused by the administered product or by other factors and there are no links between the event and the product.

Table 3 summarizes the criteria for assessing these reactions.

TABLE 3

| | |
|---|---|
| Minor | Does not interfere with the |
| | subject's normal life |
| Moderate | Slightly interferes with the |
| | subject's life |
| Serious | Significantly interferes |

### Results

The effectiveness of the treatment performed by administering the compositions of Formulation A and C according to the invention was clear and immediate. An apparent improvement of the skin texture has already been observed from the first administration. A remarkable reduction of the opacity of the treated part and an immediate lifting effect were observed.
A milder effect was observed, instead, in the group of patients who received the treatment with the chemical peeling compositions based on chlorinated derivatives of acetic acid, urea peroxide and basic buffering agent (ammonia or TEA??) after the first application and over time.
No patients treated with the compositions of Formulation A and C according to the invention reported any adverse events or major side effects after the steps of the administration process as suggested in the usual application practice, except for a mild and transient furfuraceous epidermal exfoliation, which disappeared after a few days. Photosensitization did not occur in any of the cases; the invention indeed offers the possibility of treating all phototypes throughout the entire year. The treatment was deemed comfortable and atraumatic. All subjects were able to perform their daily activities immediately after the treatment.
In some cases, in the experimental groups receiving the treatment with the compositions of Formulation B and D, white frost and skin redness appeared.
Tables 4-7 show in detail the results of the experimentation in each group.

TABLE 4
Results obtained in experimental Group A after one year from
the beginning of the trial during which each patient received two
treatment cycles, of six administrations each, six months apart.
Group A
Treatment with formulation A (TCA, propylene glycol, sodium hydroxide)

| | | | | | Side | |
|---|---|---|---|---|---|---|
| | | | Skin | Application | effects/ | |
| Subject | Age | Gender | alteration | site | reactions | Result |
| A1 | 42 | F | Non-uniform | Face | No | Excellent |
| | | | skin and | | | improvement |
| | | | cutaneous | | | |
| | | | pigmentations | | | |
| A2 | 30 | F | Stretch marks | Thigh | No | Considerable |
| | | | | | | improvement |
| A3 | 23 | M | Acne skin | Face | Transient | Improvement |
| | | | | | furfuraceous | |
| | | | | | epidermal | |
| | | | | | exfoliation | |
| A4 | 62 | M | Medium depth | Face | No | Considerable |
| | | | wrinkles | | | improvement |

TABLE 5
Results obtained in experimental Group B after one year from
the beginning of the trial during which each patient received two
treatment cycles, of six administrations each, six months apart.
Group B
Treatment with Formulation B (TCA, hydrogen peroxide, TEA)

| | | | | | Side | |
|---|---|---|---|---|---|---|
| | | | Skin | Application | effects/ | |
| Subject | Age | Gender | alteration | site | reactions | Result |
| B1 | 48 | F | Non-uniform | Face | White frost | Improvement |
| | | | skin and | | solved in a | |
| | | | cutaneous | | few days | |
| | | | pigmentations | | | |
| B2 | 29 | F | Stretch marks | Thigh | Temporary | Improvement |
| | | | | | local redness | |
| B3 | 22 | M | Acne skin | Face | Skin dryness | Improvement |
| B4 | 61 | M | Medium depth | Face | White frost | Improvement |
| | | | wrinkles | | solved in a | |
| | | | | | few days | |

TABLE 6
Results obtained in experimental Group C after one year from
the beginning of the trial during which each patient received two
treatment cycles, of six administrations each, six months apart.
Group C
Treatment with Formulation C (MCA, propylene glycol, sodium hydroxide)

| | | | | | Side | |
|---|---|---|---|---|---|---|
| | | | Skin | Application | effects/ | |
| Subject | Age | Gender | alteration | site | reactions | Result |
| C1 | 44 | F | Vaginal | Vaginal | No | Excellent |
| | | | dryness | area | | improvement |
| C2 | 53 | F | Darkening of | Labia | Minor | Considerable |
| | | | labia majora | majora and | discomfort | improvement |
| | | | and mons | mons pubis | | |
| | | | pubis | | | |
| C3 | 64 | F | Tissue | Vaginal | No | Excellent |
| | | | laxity | area | | improvement |
| C4 | 65 | F | Tissue | Perianal | No | Considerable |
| | | | laxity | area | | improvement |

TABLE 7
Results obtained in experimental Group D after one year from
the beginning of the trial during which each patient received two
treatment cycles, of six administrations each, six months apart.
Group D
Treatment with Formulation D (MCA, hydrogen peroxide, TEA)

| | | | | | Side | |
|---|---|---|---|---|---|---|
| | | | Skin | Application | effects/ | |
| Subject | Age | Gender | alteration | site | reactions | Result |
| D1 | 42 | F | Vaginal | Vaginal | Intense | No change |
| | | | dryness | area | tingling | |
| D2 | 54 | F | Darkening of | Labia | Intense | Improvement |
| | | | labia majora | majora and | reddening | |
| | | | and mons | mons pubis | | |
| | | | pubis | | | |
| D3 | 65 | F | Tissue | Vaginal | Discomfort | No change |
| | | | laxity | area | | |
| D4 | 64 | F | Tissue | Perianal | Minor redness | No change |
| | | | laxity | area | | |

The problems of instability found in the compositions of the prior art used for chemical peeling, based on mixtures of chlorinated derivatives of acetic acid, hydrogen peroxide or functional analogues, buffered with ammonia or TEA, due to the decomposition of peroxide in strongly acidic environment in water and gaseous oxygen, decomposition products and TCA not adequately buffered, produce denaturation and protein precipitation in the patients' cells and tissue by exposing them to side effects and adverse reactions, one of which is white frost. The trial conducted by performing the chemical peeling with the composition according to the invention has been shown to overcome such a technical problem by providing a very high-performing product which is useful in contrasting skim blemishes caused by stretch marks, acne scars, sun damage, skin sagging, hyperpigmentation, scars in various parts of the body, such as face, neck, bust, breast, inner thighs, arms, mons pubis, labia majora, inguinal and perianal areas.

1) A composition comprising a mixture of one or more chlorinated derivatives of acetic acid, and a glycolic solvent, buffered with a basic compound, to obtain a final pH value of the composition between 2.0 and 4.0, wherein the glycolic solvent is propylene glycol and the basic buffering agent is sodium hydroxide, for use in regenerating treatments having skin lifting and anti-age effects, for the treatment of skin imperfections.

2) The composition according to claim 1, wherein the chlorinated derivative of acetic acid is: trichloroacetic acid (TCA), dichloroacetic acid (DCA), monochloroacetic acid (MCA).

3) (canceled)

4) The composition according to claim 1 having a pH between 2.0 and 3.0.

5) The composition according to claim 1, wherein the mixture of one or more chlorinated acids of acetic acid is in a concentration ranging between 1% and 50% by weight of total composition, the glycolic solvent is in a concentration ranging between 1 and 10% by weight of total composition, the sodium hydroxide is in a concentration ranging between 1 and 10% by weight of total composition.

6) The composition according to claim 5, wherein the mixture of one or more chlorinated acids of acetic acid is in a concentration ranging between 25 and 35% by weight of total composition, the glycolic solvent is a concentration ranging between 3 and 8% by weight of total composition, the sodium hydroxide is in a concentration ranging between 3 and 8% by weight of total composition.

7) The composition according to claim 1, further comprising mandelic acid, kojic acid, coenzyme Q10, hexapeptide-1, Glycyrrhiza glabra extract and excipients.

8) The composition according to claim 1 further comprising:
a mixture of one or more chlorinated derivatives of acetic acid, except for TCA (trichloroacetic acid), in a concentration ranging between 25 and 35% by weight of total composition;
urea peroxide in a concentration ranging between 0.1 and 0.09%
glycerol in a concentration ranging between 5 and 15% by weight of total composition;
propylene glycol in a concentration ranging between 3 and 8% by weight of total composition;
mandelic acid in a concentration ranging between 3 and 8% by weight of total composition;
kojic acid in a concentration ranging between 3 and 8% by weight of total composition;
sodium hydroxide in a concentration ranging between 0.5 and 1.5% by weight of total composition;
xanthan gum in a concentration ranging between 0.25 and 2% by weight of total composition;
hexapeptide-1 in a concentration ranging between 1.5 and 3.0% by weight of total composition;
coenzyme Q10 in a concentration ranging between 0.1 and 1% by weight of total composition;
Glycyrrhiza glabra root extract in a concentration ranging between 0.1 and 1% by weight of total composition;
distilled water up to 100% by weight.

9) The composition according to claim 1 further comprising:
trichloroacetic acid 33% by weight of total composition;
glycerol in a concentration ranging between 5 and 15% by weight of total composition;
propylene glycol in a concentration ranging between 3 and 8% by weight of total composition;
mandelic acid in a concentration ranging between 3 and 8% by weight of total composition;
kojic acid in a concentration ranging between 3 and 8% by weight of total composition;
sodium hydroxide in a concentration ranging between 0.5 and 1.5% by weight of total composition;
xanthan gum in a concentration ranging between 0.25 and 2% by weight of total composition;
hexapeptide-1 in a concentration ranging between 1.5 and 3.0% by weight of total composition;
coenzyme Q10 in a concentration ranging between 0.1 and 1% by weight of total composition;
distilled water up to 100% by weight.

10) The composition according to claim 1, in a suitable form for topical administration to the skin of the subject in need of treatment.

11) The composition according to claim 10, in the form of cream, gel, ointment, liquid solution, patch.

12) A composition comprising a mixture of one or more chlorinated derivatives of acetic acid and a glycolic solvent, buffered with a basic compound, wherein the glycolic solvent is propylene glycol and the basic buffering agent is sodium hydroxide as defined in claim 1, for use in a regenerating treatment having skin lifting and anti-age effect, in the treatment of skin blemishes caused by stretch marks, acne scars, sun damage, skin sagging, hyperpigmentation, scars in various parts of the body.

13) A process of preparing the composition comprising a mixture of one or more chlorinated derivatives of acetic acid, except for trichloroacetic acid, and glycolic solvent, buffered with a basic compound, wherein the glycolic solvent is propylene glycol and the basic buffering agent is sodium hydroxide, as defined in claim 1, comprising the following steps:
a) preparing a mixture of an appropriate amount of mandelic acid, an appropriate amount of monochlorinated and/or dichlorinated derivatives of acetic acid and an appropriate amount of kojic acid in distilled water;
b) preparing a second mixture obtained by dissolving the appropriate amount of urea peroxide in glycolic solvent;
c) mixing the mixture obtained in a) with the mixture obtained in b);
d) slowly adding to the mixture obtained in c) an amount of sodium hydroxide adapted to allow the mixture achieving a final pH value between 2.0 and 4.0;
e) adding to the mixture obtained in c) all the remaining components one by one and adjusting the final volume with distilled water.

14) A process of preparing the composition comprising a mixture of trichloroacetic acid and glycolic solvent, buffered with a basic compound, wherein the glycolic solvent is propylene glycol and the basic buffering agent is sodium hydroxide, as defined in claim 9, comprising the following steps:
a) preparing a mixture of an appropriate amount of mandelic acid, an appropriate amount of trichloroacetic acid and an appropriate amount of kojic acid in water;
b) adding to the mixture obtained in a) an appropriate amount of glycolic solvent;
c) slowly adding to the mixture obtained in b) an amount of sodium hydroxide adapted to allow the mixture achieving a pH value between 2.0 and 4.0;
d) adding to the mixture obtained in c) all the remaining components one by one and adjusting the final volume with distilled water.

15) A process of using the composition comprising a mixture of one or more chlorinated derivatives of acetic acid and glycolic solvent, buffered with a basic compound, wherein the glycolic solvent is propylene glycol and the basic buffering agent is sodium hydroxide, as defined in claim 1, comprising the following step:
a) thoroughly cleansing and drying the skin of the area where the treatment is to be administered;
b) applying an amount of composition adapted to cover an area of about 5-10 cm² of skin surface and massaging until full absorption;
c) proceeding as described in b) until the whole area is treated;
d) repeating steps b) and c) up to a maximum of five times in the same treatment session in the same skin areas;
e) e) applying a nourishing and soothing cream containing sunscreen to the entire treated area.

16. The composition according to claim 2, wherein the mixture of one or more chlorinated acids of acetic acid is in a concentration ranging between 1% and 50% by weight of total composition, the glycolic solvent is in a concentration ranging between 1 and 10% by weight of total composition, the sodium hydroxide is in a concentration ranging between 1 and 10% by weight of total composition.

17. The composition according to claim 4, wherein the mixture of one or more chlorinated acids of acetic acid is in a concentration ranging between 1% and 50% by weight of total composition, the glycolic solvent is in a concentration ranging between 1 and 10% by weight of total composition, the sodium hydroxide is in a concentration ranging between 1 and 10% by weight of total composition.

18. The process of claim 13, wherein in step b) the solvent is propylene glycol, and in step d) the mixture achieves a final pH value between 2.0 and 3.0.

19. The process of claim 14, wherein in step b) the solvent is propylene glycol, and in step c) the mixture achieves a final pH value between 2.0 and 3.0.

20. The composition according to claim 2, further comprising mandelic acid, kojic acid, coenzyme Q10, hexapeptide-1, Glycyrrhiza glabra extract and excipients.

21. The composition according to claim 4, further comprising mandelic acid, kojic acid, coenzyme Q10, hexapeptide-1, Glycyrrhiza glabra extract and excipients.
